(19) United States
(12) Patent Application Publication
(10) Pub. No.: US 2022/0296756 A1
(43) Pub. Date: Sep. 22, 2022

(54) SENSOR-HEATING ELEMENT CONFIGURATIONS AND MULTI-LOOP CONTROL CYCLES FOR DRY-HEAT STERILIZERS

(71) Applicant: Integrated Medical Technologies, Inc., Bloomington, IL (US)

(72) Inventors: Nelson S. Slavik, Niles, MI (US); Michael H. Linse, Corvallis, OR (US); Gary Jay Oliver, Corvallis, OR (US); William B. Smith, Bloomington, IL (US)

(21) Appl. No.: 17/699,111

(22) Filed: Mar. 19, 2022

Related U.S. Application Data
(60) Provisional application No. 63/163,059, filed on Mar. 19, 2021.

(51) Int. Cl. A61L 2/24; A61L 2/06

(52) U.S. Cl. CPC A61L 2/24; A61L 2/06; A61L 2202/122; A61L 2202/14; A61L 2202/15

(57) ABSTRACT

Methods and systems implement a dry-heat sterilizer configured to operate at temperatures permissive of a heat-intolerant articles and heat-intolerant packaging. To effectively and evenly transmit heat throughout a dry-heat sterilizer at such temperatures, the dry-heat sterilizer furthermore provides an electronic controller coupled to multiple sensor-heating element configurations, the electronic controller configured to compute proportional-integral-derivative (PID) terms to operate a multi-loop heat control cycle including at least a first feedback loop and a second feedback loop, and configured as an asymptotic PID controller for at least one feedback loop. In this fashion, the likelihood that spot airflow temperature at particular temperature sensors will overshoot a PID setpoint is substantially reduced. This results in spot airflow temperature at the temperature sensors being sustained at the PID setpoint asymptotically, substantially improving precision of heat control cycles.

### CROSS-REFERENCE TO RELATED APPLICATION

This application claims priority to U.S. Patent Application No. 63/163,059, entitled “HIGH VELOCITY HOT AIR STERILIZER WITH NON-CONVENTIONAL, LOWERTEMPERATURE CYCLES,” filed Mar. 19, 2021, which is expressly incorporated herein by reference in its entirety.

### BACKGROUND

Dry-heat sterilizers operate by thermal destruction of microorganisms, according to a function of time and temperature in which lower temperature requires longer time to kill specified microorganisms at specified reduction levels as established by the United States Food and Drug Administration and globally. Unlike devices rated for the more limited purposes of sanitization or disinfection, and according to federal regulatory agency jurisdiction, sterilizers should achieve required reduction levels of challenge microorganisms are at a considerably higher minimum threshold. The distinction between each of these levels is considerable in the level of thermal resistance displayed by the challenge organisms required of a process and by the required reduction levels of that organism(s). Microorganisms have a thermal resistance hierarchy as described by M. Favero and W. Bond, with bacterial spores demonstrating the most resistance and lipid-containing medium sized viruses having the greatest susceptibility to heat.

Dry-heat sterilizers utilize moving air as the fluid to transfer heat to medical instruments. For example, in high velocity hot air sterilizers, air is moved at a high rate, such as 200 air changes per minute, and high velocity flowing air provides higher rate of heat conduction and greater uniformity of temperature over lower-velocity heat sterilizers. Such mechanical devices are designed to process small loads of dental, mostly orthodontic, instruments, and rely on air mixing within the sterilization chamber to minimize hot and cold areas.

Such units perform dry-heat processing at the highest temperature compatible with most dental instruments at fastest processing time possible, which gained adoption for instrument compatibility and processing times for most instruments used in the dental industry. Temperature disparities within a dry-heat sterilizer resulting from compact inner dimensions, which further limits precision of temperature control, and the general applicability of such sterilizers to most dental instruments has limited motivation for further improving their design.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 illustrates a perspective view of a dry-heat sterilizer according to example embodiments of the present disclosure.

FIG. 2 illustrates a profile view of the front of the dry-heat sterilizer of FIG. 1, indicating the relative location of a sterilization chamber according to example embodiments of the present disclosure.

FIG. 3 illustrates a cross-sectional view of the dry-heat sterilizer of FIG. 1, depicting an air handling assembly and an airflow circuit according to example embodiments of the present disclosure.

FIG. 4 illustrates a top profile view of the dry-heat sterilizer according to example embodiments of the present disclosure.

FIGS. 5A and 5B illustrate section views of upper and lower heating assemblies according to example embodiments of the present disclosure.

FIGS. 5C and 5D illustrate section views of upper and lower heating assemblies according to alternative example embodiments of the present disclosure.

FIGS. 5E and 5F illustrate section views of upper and lower heating assemblies according to further alternative example embodiments of the present disclosure.

FIG. 6 illustrates rate of change of article temperature in relation to heat transfer rate according to example embodiments of the present disclosure.

FIG. 7A illustrates spot airflow temperature at a sterilization air exhaust port over three control phases of an electronic controller configured to operate a dry-heat sterilizer from a cold start according to example of the present disclosure.

FIG. 7B illustrates spot airflow temperature at a sterilization air exhaust port over control phases of an electronic controller configured to operate a dry-heat sterilizer from a cold start according to alternate examples of the present disclosure.

FIG. 7C illustrates negligible oscillation of sustained spot airflow temperature at the sterilization air exhaust port of FIG. 7B.

FIG. 8 illustrates a sterilization cycle control system according to example embodiments of the present disclosure.

FIG. 9 illustrates an example system for implementing the processes and methods described herein for implementing a multi-loop heat control cycle.

### DETAILED DESCRIPTION

Apparatuses and methods discussed herein are directed to implementing dry-heat sterilization, and more specifically an electronic controller of a dry-heat sterilizer coupled to multiple sensor-heating element configurations, the electronic controller configured to operate a multi-loop heat control cycle including at least a first feedback loop and a second feedback loop, and configured as an asymptotic PID controller for at least one feedback loop.

Sterilizers include both freestanding and tabletop machines which are operated in or near a staffed working facility, such as a laboratory, a medical clinic, a beauty salon, and such occupational locations where various equipment, instruments, and the like (subsequently “articles,” for brevity) routinely become contaminated with microorganisms and should not be used again until returned to sterile conditions (such facilities subsequently referred to as “sterilization practicing facilities,” for brevity). For example, in a dental clinic, medical practitioners wish to sterilize dental instruments which enter a patient's mouth, such as perioprobes, explorers, forceps, curettes, scalers, mouth mirrors, and the like, after every use. In the United States, sterilization of articles in such working facilities is motivated by workplace safety and public health regulations. Even in the absence of such regulations, however, sterilization of articles is motivated by the desire to avoid introducing health hazards into the sterilization practicing facility, which would ultimately result from using unsterilized articles.

For the sake of safe handling of contaminated articles before sterilization, and maintaining sterilization of articles until they are ready for use, users of contaminated articles commonly package contaminated articles while handling them. Used, contaminated articles, rather than being handled directly, can be packaged in bags, wrappings, films, or other such protective covers which enclose the articles against outside air and contact. Users of contaminated articles can also use marked packaging to enclosed contaminated articles, so as to visually indicate the articles as contaminated. Moreover, users of contaminated articles can also use packaging to safely handle articles which are edged or pointed.

For these reasons, users of contaminated articles commonly package the contaminated articles before sterilization, and do not remove the packaging after sterilization, so that the packaged articles can be stored in a sterilized state until used again. Consequently, sterilizers and packaging can be matched so that a sterilizer can sterilize articles while stored inside of packaging, without destroying the packaging itself. By way of example, to be sterilized in a steam sterilizer, articles should be packaged using material that is both air-permeable and also impermeable to microorganisms. In contrast, to be sterilized in a high-heat sterilizer, articles cannot be packaged using polymeric material that melts at high temperatures (subsequently referred to as “heat-intolerant packaging”).

Furthermore, some equipment or instruments inherently contain polymeric material that melts at high temperatures, making them incompatible with high-heat sterilizers entirely (subsequently referred to as “heat-intolerant articles”).

Consequently, users of sterilizers are responsible for matching articles, packaging, and sterilizer mechanisms in an inter-compatible manner: composition of equipment, instruments, and the like limits packaging and sterilizer selections, and, conversely, sterilizer mechanisms limit equipment and instrument selections and packaging selections. In the event that users incur substantial expenses in procuring mismatched articles and sterilizers, users may become incentivized to clean equipment or instruments by methods that do not conform to sterilization standards, resulting in poorer hygiene practices which may lead to deleterious outcomes.

Moreover, for ready access by users of contaminated articles, sterilizers are generally installed in the same sterilization practicing facilities where those articles are used. To facilitate workflows, sterilizers are often placed at or around storage cabinets where sterilized articles are stored for later use. Their power draw contributes to the overall power consumption of an occupational workspace, and therefore contributes to the operational costs of those workspaces.

Therefore, example embodiments of the present disclosure provide a dry-heat sterilizer configured to operate at temperatures permissive of a heat-intolerant articles and heat-intolerant packaging. To effectively and evenly transmit heat throughout a dry-heat sterilizer at such temperatures, the dry-heat sterilizer furthermore provides an electronic controller coupled to multiple sensor-heating element configurations, the electronic controller configured to compute proportional-integral-derivative (PID) terms to operate a multi-loop heat control cycle including at least a first feedback loop and a second feedback loop, and configured as an asymptotic PID controller for at least one feedback loop.

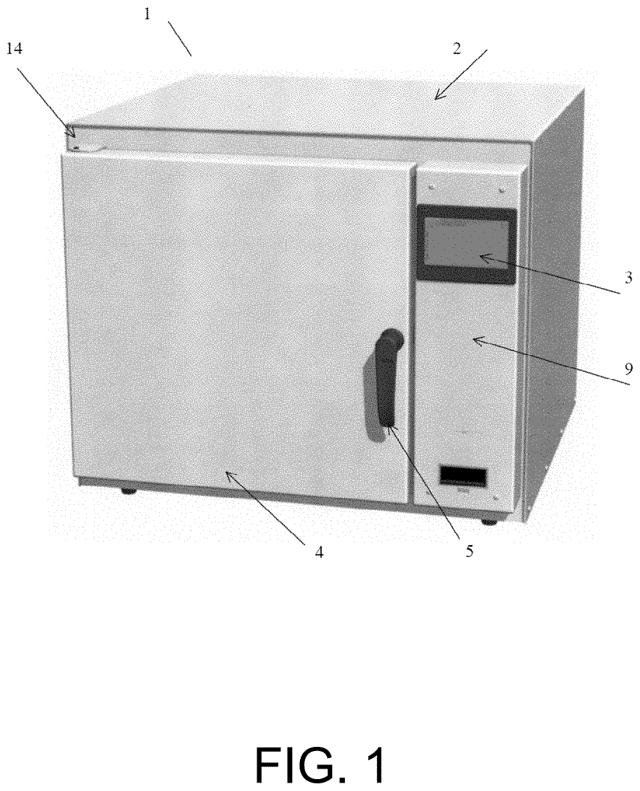

FIGS. 1 to 4 illustrate views of a dry-heat sterilizer according to example embodiments of the present disclosure. The dry-heat sterilizer 1 provides an entry door 4 at a front face, the entry door 4 being openable to receive contaminated, non-sterile articles, through an entry portal. The articles can furthermore be heat-intolerant articles, and can furthermore be packaged using heat-intolerant packaging. The dry-heat sterilizer 1 includes an outer housing 2, which can be a metal construct. The outer housing 2 establishes top, bottom and left and right sides of an inner heating chamber 11. A sterilization chamber 7 is enclosed within the heating chamber 11. The sterilization chamber 7 has a front opening 18, and an interior of the sterilization chamber 7 is accessible through the front panel 14 through the front opening 18.

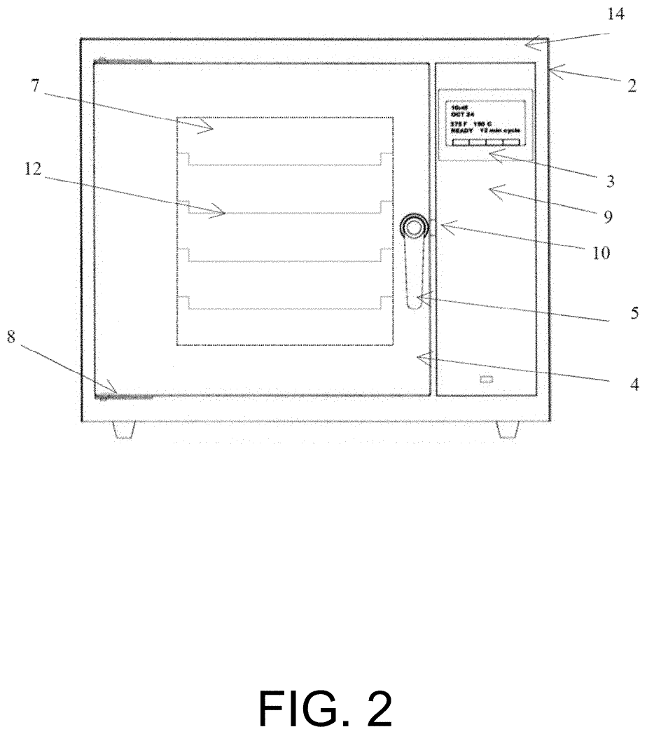

With reference to FIGS. 2, 3, and 4, the sterilization chamber 7 can be accessed for article insertion via a front entry door 4 and through an opening 16 disposed through the front panel 14 of the apparatus and through the front opening 18 of the sterilization chamber 7. Articles, including packaged articles, can be placed on perforated trays 12 which are coupled within the sterilization chamber 7 by parallel tray rails 20. The entry door 4 is attached to the front panel 14 by hinges 8 configured to allow the entry door 4 to move between an open position and a closed position. In some embodiments, the entry door 4 can be hinged vertically, while in other embodiments, the entry door 4 can be hinged horizontally.

While an electronic controller of the dry-heat sterilizer 1 is running a sterilization cycle, the entry door 4 can be configured in a closed position and locked into place by turning a locking door handle 5, disposed upon the entry door and configured to activate a door lock 10, thereby sealing the door gasket 13 tightly against the entry door 4 during the sterilization cycle to prevent outside air from entering the heating chamber 7. A controller of the dry-heat sterilizer 1 can be electronically coupled with the door lock 10, and can send a control signal to the door lock 10 to induce electronic locking (subsequently a “locking signal”). The controller can further be configured in electronic communication with control panel 9 having a keypad 3, and can receive an input signal from the control panel 9 upon a user activating the keypad 3. The controller can send a locking signal to the door lock 10 in response to the controller determining that a sterilization cycle is running, or can send a locking signal to the door lock 10 in response to the controller receiving an input signal from the control panel 9.

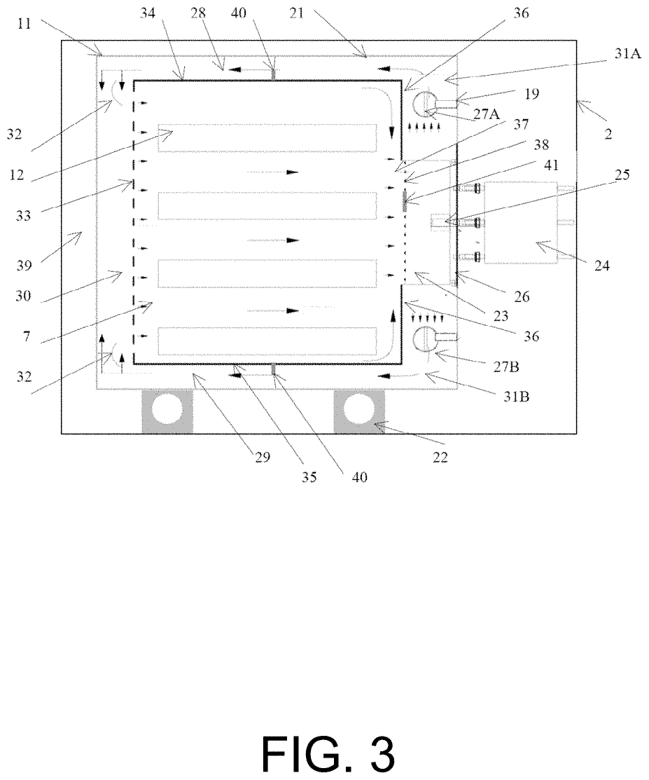

Referring to FIG. 3, a heating chamber 11 is defined by a heating chamber wall 21 which defines sides, top, and bottom of the heating chamber 11 and is constructed such as to preclude entry of outside air to the heating chamber 11, thereby allowing the heating chamber 11 and the associated air handling assembly to remain airtight when the entry door 4 is in the locked and closed position during a sterilization cycle. Encompassing the exterior of the heating chamber 11, an insulating cavity 39 is disposed between an interior face of the outer housing 2 and an exterior face of the heating chamber wall 21.

The heating chamber 11 is coupled to and supported within the outer housing 2 of the sterilizer 1 by support brackets 22 and attachment to the outer housing 2 and front panel 14 of the sterilizer 1 to form the insulation cavity 39 containing an insular material such as fiberglass. The insulation cavity 39 can minimize heat loss from the heating chamber 11 during the sterilization cycle. The insulation cavity 39 can further provide a heat barrier between the heating chamber 11 and the metal outer housing 2 of the dry-heat sterilizer 1.

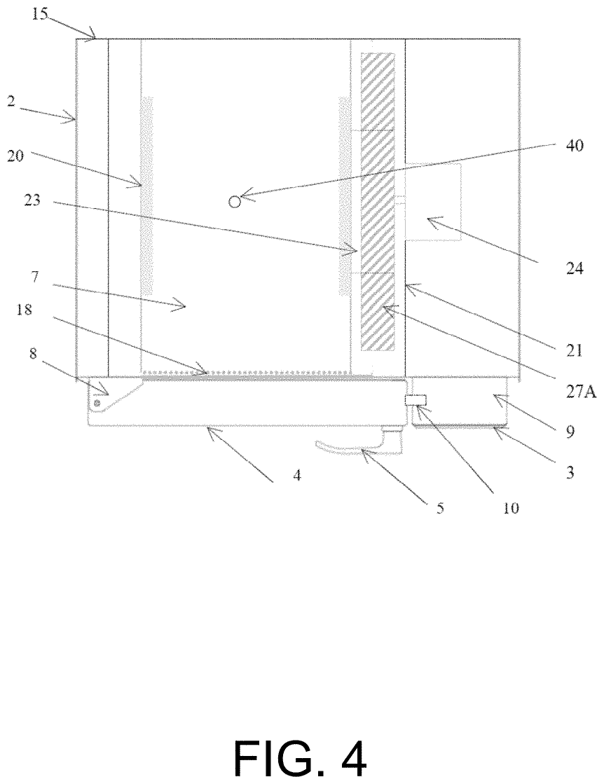

Referring to FIGS. 3 and 4, the dry-heat sterilizer 1 includes an air handling assembly which includes a circulation fan 23, an upper heating assembly 27A, a lower heating assembly 27B, dual air handling pathways, and a sterilization chamber 7. The air handling assembly propels supply air originating at the circulation fan 23 to flow through the length of the air handling assembly (this path subsequently referred to as an “airflow” at any point along the path, “spot airflow” at a particular point along the airflow defined in relation to other points along the airflow, and an “airflow circuit” in its entirety), over any number of full and partial circuits thereof.

A controller of the dry-heat sterilizer 1 can be electronically coupled with the circulation fan 23, and can send a control signal to the circulation fan 23 to power or depower the circulation fan 23, or increase power, sustain power, or decrease power to the circulation fan 23 (subsequently a “fan signal,” which should be understood as a type of control signal).

The dual air handling pathway is formed by at least two air handling pathways configured to span above and below the sterilization chamber 7 through an upper supply air duct 28 and lower supply air duct 29. The upper supply air duct 28 and the lower supply air duct 29 are respectively formed by an external face of a top wall 34 and an external face of a bottom wall 35 of the sterilization chamber 7, each in conjunction with an internal face of the heating chamber wall 21.

A first supply air duct 30 is formed by an internal face of the heating chamber wall 21 and an external face of a perforated supply air wall 33 of the sterilization chamber 7. A second supply air duct houses a circulation fan 23 and upper and lower heating assemblies 27A and 27B, and is formed by an interior face of the heating chamber wall 21 and an exterior exhaust wall 36 of the sterilization chamber 7. The exhaust wall 36 contains an exhaust portal 37 allowing airflow passage through to the circulation fan 23. The exhaust portal 37 may be covered by a mesh screen 38. A motor 24, located externally to the heating chamber 11, is connected to the circulation fan 23 by a motor shaft 25, which drives the circulation fan 23. A seal 26 is further disposed around the motor shaft 25 and upon an interior face of a heating chamber wall 21, the seal 26 configured to prevent air infiltration into the heating chamber 11 by way of the motor shaft 25.

Referring to FIG. 3, the air handling assembly configures the circulation fan 23 to create positive pressure and air velocity upon supply airflow at the circulation fan 23, propelling the supply airflow bi-directionally through an upper branch 31A and a lower branch 31B of the second supply air duct.

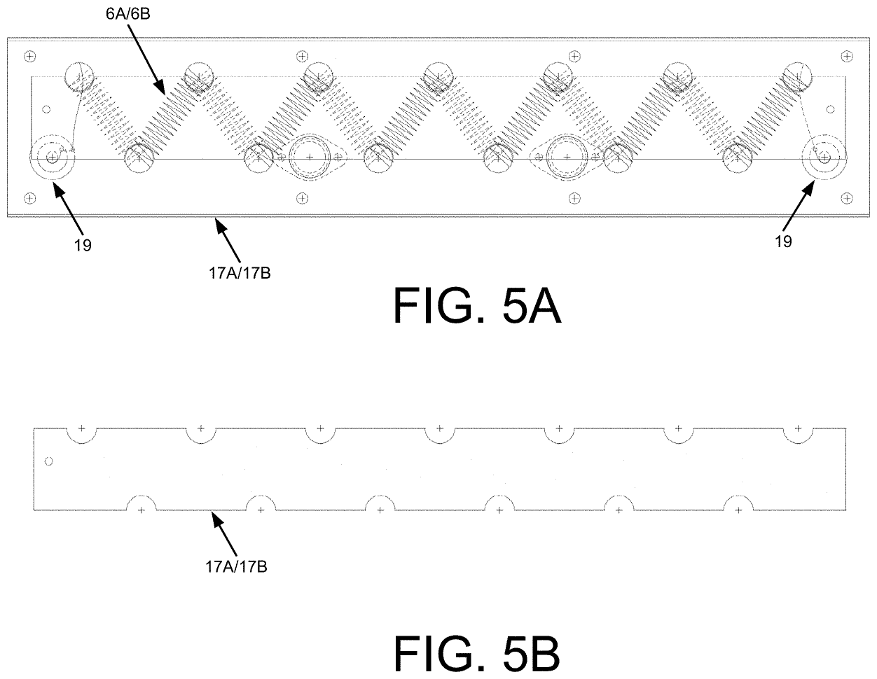

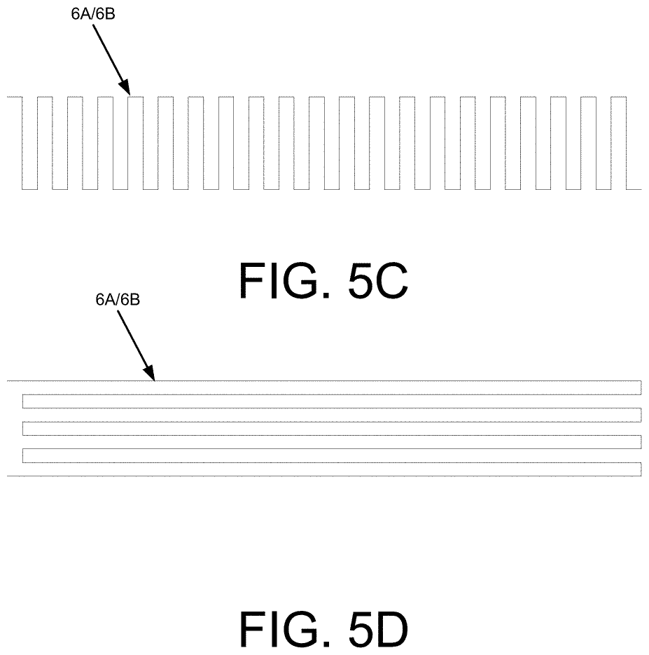

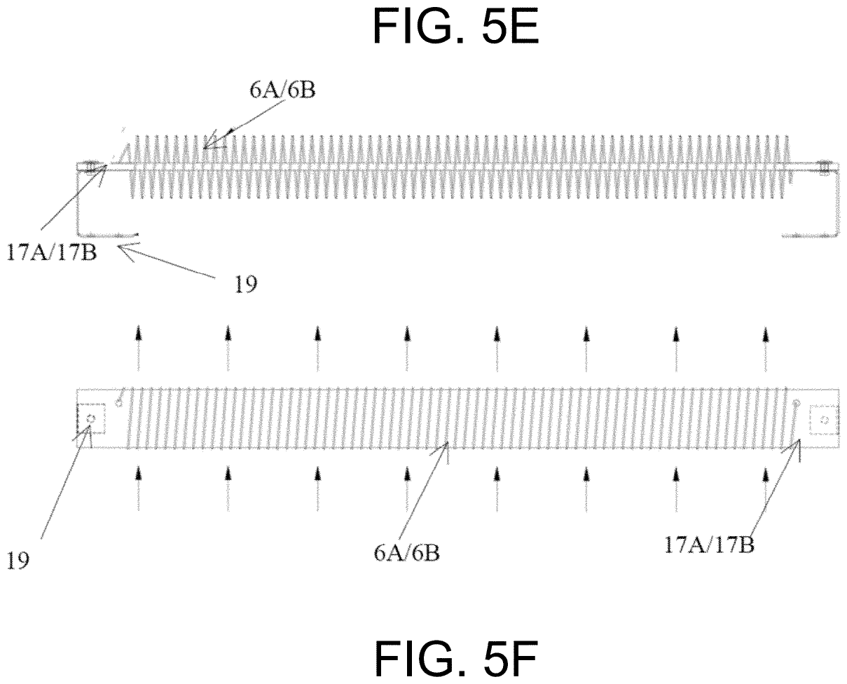

According to example embodiments of the present disclosure, the upper branch 31A and the lower branch 31B respectively contain an upper heating assembly 27A and a lower heating assembly 27B, each heating assembly comprising an openwork heating element. An “openwork heating element” according to example embodiments of the present disclosure can encompass any among a variety of differently constructed heating elements. For illustrative purposes, several examples of openwork heating elements shall be described subsequently with reference to FIGS. 5A through 5F, but it should be understood that these examples shall not be limiting as to the scope of the present disclosure.

FIGS. 5A and 5B illustrate section views of upper and lower heating assemblies 27A or 27B according to example embodiments of the present disclosure. Each of the heating assemblies 27A or 27B can include a respective coiled heating element 6A and 6B, each of the heating elements 6A and 6B coiled again to span the length of an electrical insulator 17A and 17B, respectively, and attached to an interior face of the heating chamber wall 21 by a mounting support 19 at each end of each heating assembly 27A and 27B. Thus, the coiled heating elements 6A and 6B can be subsequently described as each having a “coiled-coil,” and can be subsequently described as each having “first-order” coiling (referring to the coiling of the heating element wire), and furthermore each having “second-order” coiling (referring to the further coiling of the coiled wire). A coiled-coil heating element should be understood as an example of an openwork heating element according to example embodiments of the present disclosure.

A controller of the dry-heat sterilizer 1 can be electronically coupled with the coiled heating elements 6A and 6B, and can send a control signal to the coiled heating elements 6A and 6B to induce increased or decreased power draw (subsequently a “power signal,” which should be understood as a type of control signal).

The heating assemblies 27A and 27B are positioned immediately above and below the circulation fan 23 and sized to enhance heat dissipation to the bifurcated airflows propelled by the circulation fan 23. By way of example, each heating element 6A and 6B, which can be a wire manufactured from any heat-conducting metal, has a wire diameter and is first-order coiled to a coil diameter (then second-order coiled about the electrical insulators 17A or 17B).

Cross-sectional (orthogonal to an airflow direction) inner dimensions of the upper branch 31A and the lower branch 31B of the second supply air duct each include a respective width and a respective depth, a width being defined herein as the cross-sectional inner dimension of the upper branch 31A and the lower branch 31B along the exhaust wall 36, and a depth being defined herein as the cross-sectional inner dimension of the upper branch 31A and the lower branch 31B orthogonal to the exhaust wall 36. The width can be substantially longer than the depth. For example, according to one possible example embodiment of the present disclosure, the width can be approximately 14 inches and the depth can be approximately 1 inch.

It should be understood that the cross-sectional width and the cross-sectional depth as described above do not limit a cross-section of the upper branch 31A or the lower branch 31B to being rectangular, and the cross-sectional inner dimensions can describe an approximate width and an approximate depth of a second supply air duct which is oblong in cross-section, trapezoidal in cross-section, or any other shape substantially longer in one dimension than a second dimension.

It should be understood that an electrical insulator 17A or 17B, which can be composed of any structurally rigid electrically insulating material, supports heating elements 6A and 6B along a width of the upper branch 31A and the lower branch 31B, and can bring each heating element 6A and 6B into proximity to inner walls of the second supply air duct (such as the exhaust wall 36), leaving narrow spacing from the electrical insulator 17A and 17B to the inner walls so that bifurcated airflows through the upper branch 31A and the lower branch 31B is substantially funneled over heating elements 6A and 6B, causing each bifurcated airflow to substantially evenly absorb heat dissipated from the coiled heating elements 6A and 6B.

It should further be understood that the electrical insulator 17A or 17B, as illustrated in FIG. 5B, can have coiled grooves cut across its surface wherein a heating element 6A or 6B can be mounted and held in place.

Moreover, the electrical insulators 17A and 17B, respectively, hold the heating elements 6A and 6B with their length oriented laterally relative to airflow, while the heating elements are coiled establishing intra-coil openings permitting airflow within the first-order coils, while each coil of the heating element is further coiled establishing inter-coil openings permitting airflow between the second-order coils (while airflow is not permitted within the second-order coils, which wrap directly around the electrical insulator 17A or 17B). In this configuration, quantity of air exposed to heat dissipated by the heating elements can be enhanced, thereby minimizing air temperature extremes or differentials occurring between heated and unheated or minimally heated air. Placing heating elements proximal to the circulation fan situated within the plenum allows maximum heat dissipation to airflow over and under the heating elements. Similarly, sizing of the heating elements to fit across the entire plenum width can minimize the amount of airflow around the ends of the heating elements, and further enhance heat dissipation to airflow over, under, between, and through the coils of the heating elements.

The wire diameter and the coil diameter can be configured to minimize heat dissipation from a heating element 6A or 6B to the electrical insulator 17A or 17B (resulting from contact between the heating element 6A or 6B and the electrical insulator 17A or 17B), and, in turn, enhance heat dissipation from the heating element 6A or 6B into each bifurcated airflow over the heating element 6A or 6B. By way of example, a larger coil diameter can result in each first-order coil of the heating element 6A or 6B contacting the electrical insulator 17A or 17B minimally on one side, while the rest of each first-order coil of the heating element 6A or 6B, on all sides, are exposed to airflow. Moreover, a smaller wire diameter can result in greater ease of heat dissipation, without being so small as to compromise durability and lifespan of a heating element 6A and 6B.

By way of example, without limitation, given a heating element wire having surface load of approximately 7 W per square centimeter, total wire length can be approximately 25-30 times the cross-sectional width of the upper branch 31A and the lower branch 31B, coil diameter can be approximately 0.4 to 0.6 inches, and wire diameter can be approximately 10 to 15 times narrower than the coil diameter, these dimensions collectively illustrating an example of heating element dimensions which can improve heat dissipation and minimize mass while achieving greater durability and lifespan.

FIGS. 5C and 5D illustrate section views of upper and lower heating assemblies 27A or 27B according to alternative embodiments of the present disclosure. Each of the heating assemblies 27A or 27B can include a respective zigzag heating element 6A and 6B, composed of a wire manufactured from any heat-conducting metal constructed to span a width and depth of a second supply air duct without support by an electrical insulator. FIG. 5C illustrates a heating element constructed to zigzag widthwise. FIG. 5D illustrates a heating element constructed to zigzag depthwise. A zigzag heating element should be understood as an example of an openwork heating element according to example embodiments of the present disclosure.

In the examples of FIG. 5C and FIG. 5D, the zigzagging heating element wire can be mounted to inner walls of the second supply air duct at any number of its turns. Furthermore, in the example of FIG. 5C, the widthwise zigzagging heating element wire can be mounted to inner walls of the second supply air duct between its turns, so as to avoid long spans of wire hanging unsupported.

A controller of the dry-heat sterilizer 1 can be electronically coupled with the zigzag heating elements 6A and 6B, and can send a power signal to the zigzag heating elements 6A and 6B to induce increased or decreased power draw.

The heating assemblies 27A and 27B are positioned immediately above and below the circulation fan 23 and sized to enhance heat dissipation to the bifurcated airflows propelled by the circulation fan 23. By way of example, each zigzag heating element 6A and 6B, which can be a wire manufactured from any heat-conducting metal, can substantially occupy the cross-sectional inner dimension of the upper branch 31A and the lower branch 31B orthogonal to the exhaust wall 36, leaving narrow spacing from the zigzag heating element 6A and 6B to the inner walls, as well as through gaps in the zigzag heating element 6A and 6B, so that bifurcated airflows through the upper branch 31A and the lower branch 31B is substantially funneled over zigzag heating elements 6A and 6B, causing each bifurcated airflow to substantially evenly absorb heat dissipated from the zigzag heating elements 6A and 6B.

FIGS. 5E and 5F illustrate section views of upper and lower heating assemblies 27A or 27B according to example embodiments of the present disclosure. Each of the heating assemblies 27A or 27B can include a respective coiled heating element 6A and 6B spanning the length of a respective electrical insulator 17A and 17B, each of the heating elements 6A and 6B being coiled only to the first order, and the respective electrical insulator 17A and 17B being attached to an interior face of the heating chamber wall 21 by a mounting support 19 at each end of each heating assembly 27A and 27B. Thus, the coiled heating elements 6A and 6B can be subsequently described as each having first-order coiling but not second-order coiling. A coiled heating element should be understood as an example of an openwork heating element according to example embodiments of the present disclosure.

A controller of the dry-heat sterilizer 1 can be electronically coupled with the coiled heating elements 6A and 6B, and can send a power signal to the coiled heating elements 6A and 6B to induce increased or decreased power draw.

The heating assemblies 27A and 27B are positioned immediately above and below the circulation fan 23 and sized to enhance heat dissipation to the bifurcated airflows propelled by the circulation fan 23. By way of example, each heating element 6A and 6B, which can be a wire manufactured from any heat-conducting metal, has a wire diameter and is first-order coiled to a coil diameter about the electrical insulators 17A or 17B.

It should be understood that an electrical insulator 17A or 17B, which can be composed of any structurally rigid electrically insulating material, supports heating elements 6A and 6B along a width of the upper branch 31A and the lower branch 31B, and can bring each heating element 6A and 6B into proximity to inner walls of the second supply air duct (such as the exhaust wall 36), leaving narrow spacing from the electrical insulator 17A and 17B to the inner walls so that bifurcated airflows through the upper branch 31A and the lower branch 31B is substantially funneled over heating elements 6A and 6B, causing each bifurcated airflow to substantially evenly absorb heat dissipated from the coiled heating elements 6A and 6B.

Moreover, the electrical insulators 17A and 17B, respectively, hold the heating elements 6A and 6B with their length oriented laterally relative to airflow, while the heating elements are coiled substantially parallel to a direction of airflow. In this configuration, quantity of air exposed to heat dissipated by the heating elements can be enhanced, thereby minimizing air temperature extremes or differentials occurring between heated and unheated or minimally heated air. Placing heating elements proximal to the circulation fan situated within the plenum allows maximum heat dissipation to airflow over and under the heating elements. Similarly, sizing of the heating elements to fit across the entire plenum width can minimize the amount of airflow around the ends of the heating elements, and further enhance heat dissipation to airflow over and under the coils of the heating elements.

The wire diameter and the coil diameter can be configured to minimize heat dissipation from a heating element 6A or 6B to the electrical insulator 17A or 17B (resulting from contact between the heating element 6A or 6B and the electrical insulator 17A or 17B), and, in turn, enhance heat dissipation from the heating element 6A or 6B into each bifurcated airflow over the heating element 6A or 6B. By way of example, a larger coil diameter can permit substantial airflow between each first-order coil of the heating element 6A or 6B and the electrical insulator 17A or 17B, so that each first-order coil of the heating element 6A or 6B, on all sides, is substantially exposed to airflow. Moreover, a smaller wire diameter can result in greater ease of heat dissipation, without being so small as to compromise durability and lifespan of a heating element 6A and 6B.

By way of example, without limitation, coil diameter can be approximately 0.8 to 1.2 inches, and wire diameter can be approximately 45 times narrower than the coil diameter, these dimensions collectively illustrating an example of heating element dimensions which can improve heat dissipation and minimize mass while achieving greater durability and lifespan.

By way of the above-listed examples of openwork heating elements, it may be appreciated that an “openwork heating element” describes a variety of manufactures, which may be self-supporting or may be mounted across a supporting structure such as an electrical insulator. Openwork heating elements can be manufactured from, as described above, any heat-conducting metal in a variety of rigid two-dimensional openworks or rigid three-dimensional openworks, whether constructed additively by combining rigid or semi-rigid pieces of material, or constructed subtractively by cutting openings in a solid piece of material. Rigid two-dimensional openworks or rigid three-dimensional openworks constructed in these manners can include coils of various dimensions, orders, and numbers; weaves of intersecting wires or rigid strands; fences; rails; latticeworks; cages; baskets; and any other such openwork constructed from heat-conducting metal having openings permitting airflow therethrough.

Thus, according to any example embodiments of the present disclosure as described above or as provided by the scope of the present disclosure, the circulation fan 23 propels each bifurcated airflow laterally over the heating assemblies 27A and 27B, respectively, to enhance air exposure to heat dissipation from each heating element 6A and 6B, by airflow through any number of openings of the openwork heating elements. The circulation fan 23 further propels each heated, bifurcated airflow into the upper supply air duct 28 and the lower supply air duct 29, each supply air duct having a substantially angular turn which directs airflow past, respectively, a temperature sensor 40A and a temperature sensor 40B located, respectively, partway down the length of and across both the upper supply air duct 28 and lower supply air duct 29. Temperature sensors according to example embodiments of the present disclosure can be, for example, thermocouples.

Each temperature sensor 40A and 40B is electronically coupled to a controller, and configured to relay temperature measurements proximate to, and downstream in airflow from, the heating elements 6A and 6B, which the controller uses as input to send power signals to power or depower the heating elements 6A and 6B, as shall be described subsequently, to thereby minimize air temperature deviations after exposure to the upper and lower heating elements 27A and 27B. It should be understood that each temperature sensor 40A and 40B should be set downstream and apart from the heating elements 6A and 6B, respectively, while remaining in proximity of the respective heating elements so that airflow minimally drops in temperature as it passes from a heating element to the respective temperature sensor.

Each heated, bifurcated airflow then moves through the upper supply air duct 28 and the lower supply air duct 29, rejoining each other and entering the first supply air duct 30, the supply air duct having a substantially angular turn formed in its path.

The heated, rejoined airflow then enters the sterilization chamber 7 through some number of perforations in the perforated supply air wall 33 of the sterilization chamber 7. To assist in uniform distribution of airflow to the sterilization chamber 7, any number of airflow diverters 32 may be disposed at junctions of each of the upper supply air duct 28 and lower supply air duct 29 of the first supply air duct 30, to evenly direct air across the perforated supply air wall 33. Dependent on the need for additional airflow distribution to the sterilization chamber 7, other airflow diverters 32 may also be disposed in other locations throughout the airflow pathway.

The heated, rejoined airflow, which can now be substantially uniform in temperature, can enter the sterilization chamber 7 through the perforated supply air wall 33 traveling horizontally across a width of the sterilization chamber 7 toward the air exhaust wall 36 as directed by negative pressure induced by the circulation fan 23. Airflow is pulled from the sterilization chamber 7 through the air exhaust portal 37, completing a circuit from the originating point mentioned above, and reenters the circulation fan 23. A screen 38 is coupled to and spans the air exhaust portal 37, configured to protect the circulation fan 23 from loose objects that may enter from the sterilization chamber 7.

Dry-heat sterilization is based on heat conduction to achieve a temperature required to initiate bacterial spore kill in contaminated articles. Conventionally, dry-heat sterilizers have included static or passive hot air sterilizers, in which air convection is generated solely by gravity as hot air rises and cooler air descends; mechanical low velocity forced-convection sterilizers, in which heated air is moved at 10 to 20 air changes per minute which serves to minimize hot and cold areas within the chamber; and high velocity hot air sterilizers, in which air is moved at a high rate, such as 200 air changes per minute, in which high velocity flowing air amplifies the rate of heat conduction and improves uniformity of temperature.

Each of these conventional dry-heat sterilizers includes various trade-offs in functionality and operating parameters inherent in its design. Both static air and low velocity mechanical convection sterilizers require minimally one hour (at 340° F. and 360° F.) or two hours (at 320° F.) to achieve sterilization. These sterilizers are static, passive units that relied on gravity flow of heat for temperature dispersal. As such, chamber temperatures were non-uniform, generating cool and warm areas throughout the sterilization chamber. Since the air motion is only generated from hot air rising and colder air falling, heat conduction to the instrument is a slow process and requires a minimum of an hour at 340 and 360° F. and two hours at 320° F. Temperature control is marginal in such systems and temperatures far below and far above the set sterilization temperature are typical. These limitations are detrimental to widespread adaptation of a static dry-heat process as an instrument sterilization technology.

In contrast, high velocity hot air sterilizers can sterilize in six to twelve minutes (at 375° F.), depending on instrument type or packaging. Such units perform dry-heat processing by reaching the highest temperature compatible with heat-tolerant dental instruments in a short time, but, consequently, are not compatible with heat-intolerant articles and heat-intolerant packaging.

Whereas other sterilization techniques operate at lower temperatures, such as non-thermal, chemical agent sterilization technologies or submersion in disinfecting chemical solutions, technologies using gaseous, vaporous, or liquid sterilants are substantially reduced in effectiveness due to inability of chemical agents to contact all instrument surfaces with the required contact time and agent concentration, and potential for reduced chemical agent effectiveness due to organic residues or other interfering materials. Such techniques are also incompatible with a variety of packaging.

Therefore, a dry-heat sterilizer according to example embodiments of the present disclosure is configured to reach and maintain sterilization temperatures which are compatible with heat-intolerant articles and heat-intolerant packaging, while still arriving at spore inactivation thresholds within a substantially short time.

Example embodiments of the present disclosure provide an electronic controller configured to modulate air velocity to and through the sterilization chamber, by a multi-loop heat control cycle including at least a first feedback loop and a second feedback loop, and configured as an asymptotic PID controller for at least one feedback loop, which enables maintaining a substantially uniform and accurately controlled sterilization temperature within the sterilization chamber. A dry-heat sterilizer according to example embodiments of the present disclosure can perform dry-heat sterilization cycles at temperatures between 240 and 320° F., integrating temperature control and air velocity to modulate rates of temperature rise during the instrument warm-up phase of sterilization process and for instrument temperature maintenance during the remaining time.

According to example embodiments of the present disclosure, heated, high velocity dry air serves as a sustained-temperature heat source, conducting heat directly to contaminated articles. For that air temperature to remain sustained and to maintain maximum heat conduction efficacy before a sterilization cycle begins, the heat of that air should be replenished to replace the heat conducted to contaminated articles. Thus, example embodiments of the present disclosure provide a closed air handling system that rejuvenates heat-expended air by a circulation fan and heating elements, further providing a heat source which can be regulated indefinitely and uniformly by a controller as shall be subsequently described.

Equation 1 below describes heat conduction to an article from air, which is directly related to air velocity as defined in the Mass Flow Rate (m) comprised of air density and volume flow rate.

Q_{Air}=\dot{m}c(ΔT)
Where: Q_{Air}=Heat Conduction in Joules (J) of the Air;
m=Mass Flow Rate=Volume Flow Rate of Air times the Density of Air;
c=Specific Heat in J/Kg° C. of Air; and
ΔT=Difference in Temperature in ° C. between heated air and article.

As seen in Equation 1, as the flow rate increases, an increase in heat transfer follows. Increasing airflow velocity is function of fan control which can be regulated by a controller, as shall be described subsequently.

Heat conduction to articles in the sterilizer according to example embodiments of the present disclosure is additionally subject to mass of articles and specific heat constant of the article's material composition. The mass and specific heat of articles can be understood as remaining unchanged through the sterilization process, while difference in temperature between the article and air diminishes over a duration of the sterilization process. This is because, inside a sterilization chamber, heat is transferred from heated air to comparatively cooler articles, and transfer of heat can continue at non-negligible rates while there is a substantial difference in temperature between the air and the article.

After the two reach approximately the same temperature, thermal equilibrium is substantially established and heat transfer becomes negligible. In a dry-heat sterilizer according to example embodiments of the present disclosure, heat is replenished to air over time, so equilibrium is reached by rising temperature of articles, not falling temperature of air.

In Equation 2 below, the difference in temperature between the article and air is variable, with article temperature increase also being variable while holding the temperature of air in a sterilizer chamber substantially sustained before a sterilization cycle begins.

Q_{Article}=mc(ΔT)
Where: Q_{Article}=Heat Conduction in Joules (J) of the Article
m=Mass in Kgs;
c=Specific Heat in J/Kg° C. of the Article; and
ΔT=Difference in Temperature in ° C. between Heated Air and Article.

The controller according to example embodiments of the present disclosure is configured to account for effects of air velocity and heat applied to replenish air heat absorbed by articles, thereby accurately regulating rates of temperature increase and sustaining temperature through durations of designated low-temperature cycles, achieving efficient sterilization cycle times.

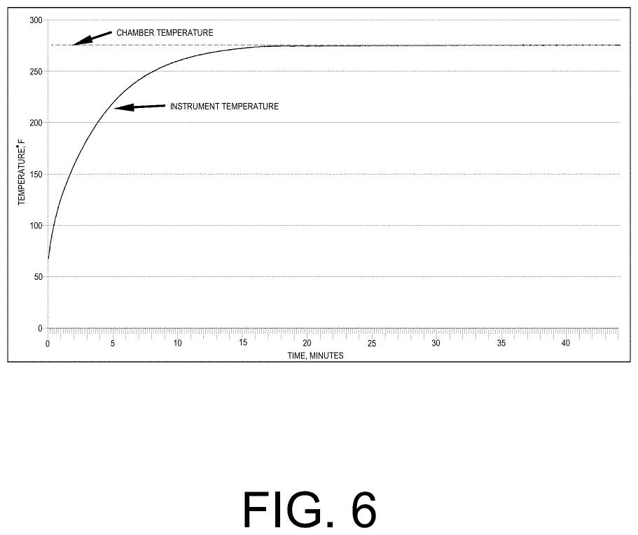

Furthermore, according to Equation 2 and illustrated in FIG. 6, the rate at which temperature changes is proportional to the rate at which heat is transferred. The temperature of an article changes more rapidly while heat is transferred at a high rate and less rapidly while heat is transferred at a low rate.

In FIG. 6, the substantially sustained air temperature in a sterilization chamber is illustrated as a broken horizontal line, while article temperature is illustrated as a solid line. The vertical axis represents temperature (assuming already sustained in the sterilization chamber over contaminated articles) in degrees Fahrenheit, and the horizontal axis represents time in minutes (i.e., since temperature became sustained). The slope of the article temperature is a measure of the rate of heat transfer. Over time, rate of heat transfer decreases. Initially heat is being transferred at a high rate as reflected by the steeper slopes, and as time progresses the slope of the article temperature line decreases.

The variable that contributes to the rate of conduction is affected by the temperature difference between the cooler article and the hotter air. As the articles warm, the temperature difference decreases, so the rate of temperature increase decreases. As the temperature difference (distance between the two lines) asymptotically approaches zero, the rate of heat transfer also approaches zero.

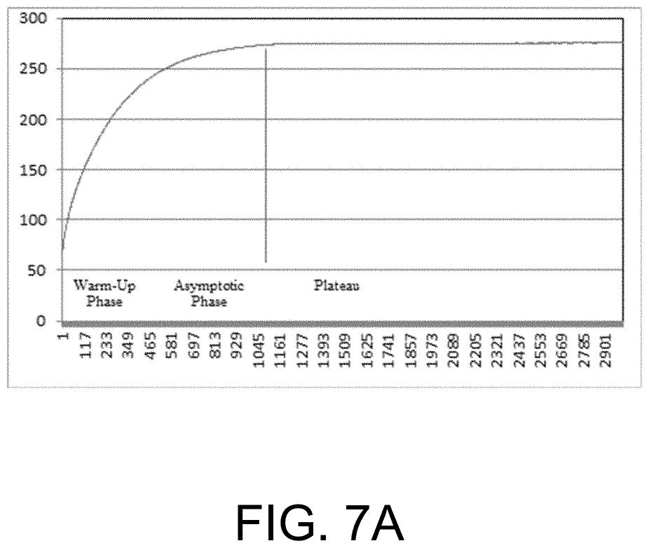

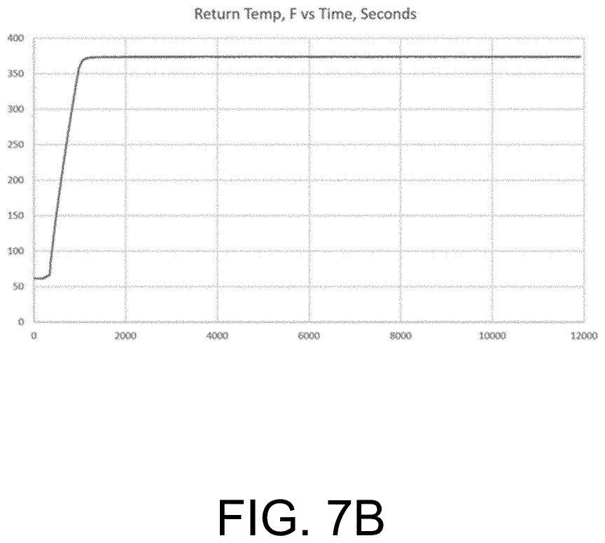

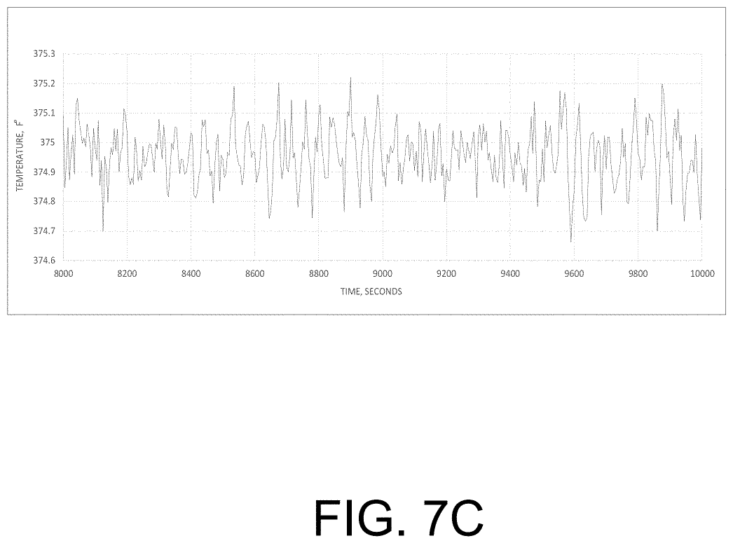

Referring to FIGS. 6 and 7, the time required to heat an article is a function of sterilization chamber temperature and mass of the article. Control of temperature occurs at three phases and is measured at least in part by the sterilization cycle control temperature sensor 41 located within the sterilization chamber 7 approximately at the exhaust port screen 38.

FIG. 7A illustrates spot airflow temperature at a sterilization air exhaust port over three control phases of an electronic controller configured to operate a dry-heat sterilizer from a cold start according to examples of the present disclosure. In this example, heating elements 6A and 6B are coiled-coil heating elements. The vertical axis represents temperature (not yet sustained, and to be sustained, in the sterilization chamber over contaminated articles) in degrees Fahrenheit, and the horizontal axis represents time in seconds. The three phases are approximately delineated by vertical lines. The controller operates in a first phase occurs during an initial stage of article warmup, in which the controller computes a first PID feedback loop to keeps the heating elements 6A and 6B working at an upper range of power. At approximately 30-35° F. below a target sterilization temperature, the controller operates in a second temperature control phase, in which the controller computes a second PID feedback loop to reduce power to the heating elements, reducing the rate of temperature increase as article temperature asymptotically approaches the target temperature.

After the target temperature is approximately attained and article temperature has approximately plateaued, a third control phase introduces air velocity modulation by controlling fan function to stabilize the temperature from any minor temperature increases or decreases that may occur during the remaining sterilization cycle duration. By way of example, low-temperature sterilization cycles according to example embodiments of the present disclosure can extend to approximately 4.5 hours at approximately 270° F.

The sterilization cycle control temperature sensor 41 also measures temperature of spot airflow, which has passed over contaminated articles, where it enters the sterilization air exhaust port 37 to quantify heat desired for supply air re-heating and subsequent re-circulation as desired through the duration of a sterilization cycle. Thus, the temperature sensor 41 is upstream of the circulation fan 23 in an airflow circuit, and the airflow circuit passes through the sterilization chamber 7 after passing the temperature sensors 40A and 40B and before reaching the temperature sensor 41. The sterilization cycle control temperature sensor 41 is electronically coupled to a controller, and configured to relay temperature measurements, which the controller uses as input to send power signals to power or depower the heating elements 6A and 6B, or increase power, sustain power, or decrease power to the heating elements 6A and 6B, as shall be described subsequently.

Similarly, FIG. 7B illustrates spot airflow temperature at a sterilization air exhaust port over control phases of an electronic controller configured to operate a dry-heat sterilizer from a cold start according to alternate examples of the present disclosure. In this example, heating elements 6A and 6B are coiled heating elements.

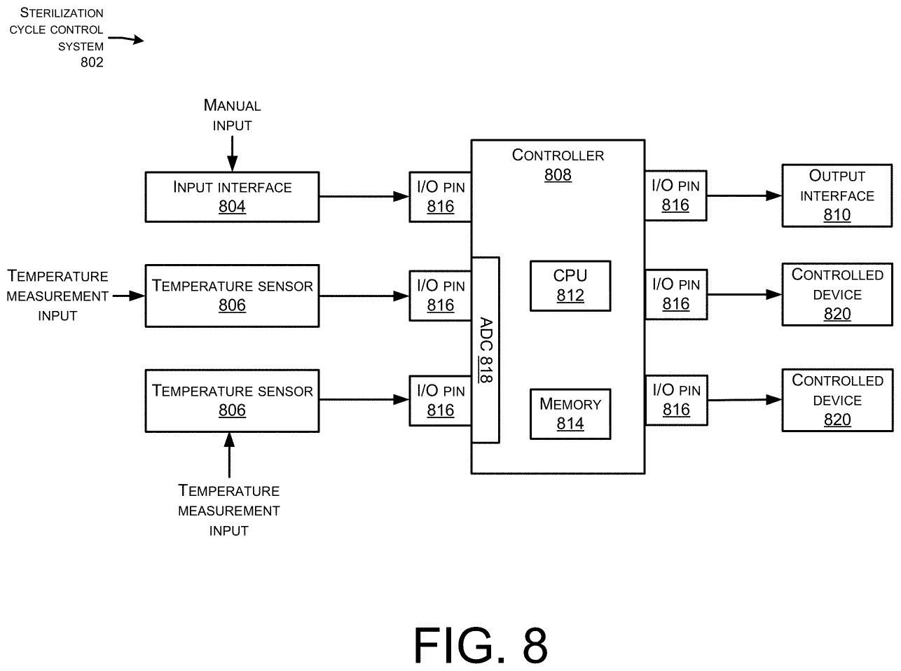

FIG. 8 illustrates a sterilization cycle control system 802 according to example embodiments of the present disclosure. A sterilization cycle control system 802, according to example embodiments of the present disclosure, may include one or more sets of computer-readable instructions executable by a controller to perform tasks that include collecting an input from multiple input sources, the input including parameters and measurements; computing input parameters and measurements according to one or more feedback loops described by the computer-readable instructions to derive multiple control signals; and outputting control signals to multiple controlled devices 820, including at least a fan signal to a circulation fan and a power signal to a heating element.

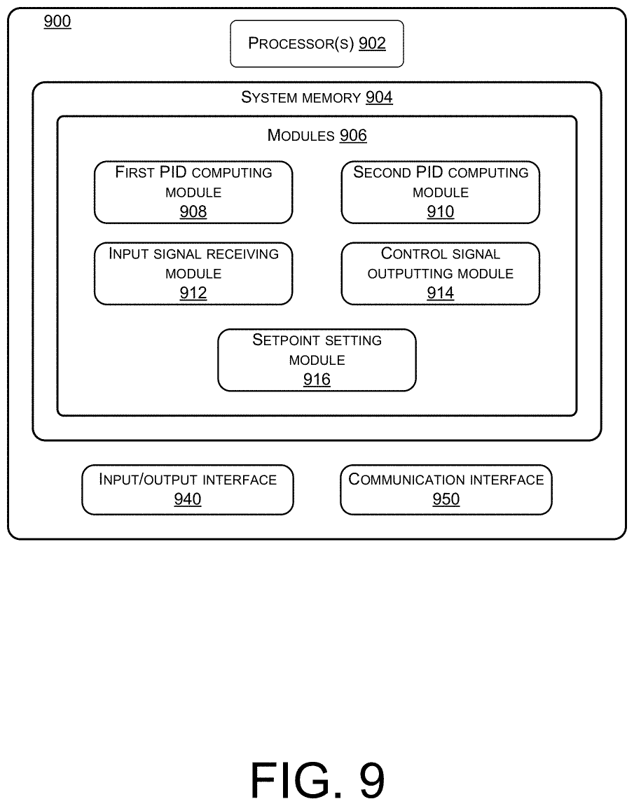

A sterilization cycle control system 802 may be a computing system integrated into the dry-heat sterilizer 1 of FIG. 1, or may be a separate computing system electronically coupled to the dry-heat sterilizer 1 of FIG. 1, where examples of a computing system according to example embodiments of the present disclosure as shall be described subsequently with reference to FIG. 9. Without reiterating the subsequent description, it should be understood, for the purpose of understanding example embodiments of the present disclosure, that the sterilization cycle control system 802 may include various input interfaces and input devices, including one or more input interface(s) 804 and multiple temperature sensors 806. Each of the various input interfaces and temperature sensors may be in communication with a controller 808 of the sterilization cycle control system 802 over a communication bus. The sterilization cycle control system 802 may further include multiple output interfaces 810.

For example, an input interface 804 can be a keypad 3 as described above with reference to FIG. 1, operable by a user to input one or more parameters which are converted by an integrated circuit of a control panel 9 into an input signal sent to the controller 808, as shall be described subsequently. An output interface 810 can be a display screen of the control panel 9, configured to display one or more parameters input by a user as determined by an integrated circuit of the control panel 9.

The controller 808 may be configured to process input from each of the input interfaces and temperature sensors, including a temperature setting input from one or more input interface(s) 804; and temperature measurement input from multiple temperature sensors 806.

The controller 808 can be configured as a PID controller using a temperature setting as a setpoint of a feedback loop. A setpoint, in the context of a PID controller, should be understood as a desired value of a variable, the PID controller being configured to substantially sustain that value of the variable in a feedback loop by sending control signals to one or more controlled devices 820 and obtaining feedback variable values from further input signals. According to example embodiments of the present disclosure, a setpoint can be a temperature setpoint, and a setpoint input by a user can be a temperature value that the user desires to configure the PID controller to substantially sustain before a sterilization cycle begins in the dry-heat sterilizer 1. According to example embodiments of the present disclosure, the controller 808 can be configured as a PID controller which can substantially sustain the temperature measurement at approximately the value of the temperature setpoint (to within approximately 1-2° F.) by sending power signals to heating elements 6A and 6B and a circulation fan 23 as described above with reference to FIG. 1, and which can obtain feedback variable values from the temperature sensors 40A, 40B, and 41 as described above with reference to FIG. 1.

However, according to example embodiments of the present disclosure, not all setpoints are set by user input. A controller 808 is further configured to derive an offset setpoint from an input setpoint, where the offset setpoint is slightly higher or slightly lower (by, for example, approximately 1-2° F.) in temperature value than the input setpoint. Moreover, a controller 808 is configured to modify a setpoint based on feedback from one or more feedback loops. This shall be described subsequently.

The controller 808 can be configured as a PID controller by the one or more sets of computer-readable instructions stored on a computer-readable storage medium. By way of example, the controller 808 can be a universal process controller or a programmable process controller, each being an example of a microcontroller, i.e., an integrated circuit composed of at least one or more central processing units (CPUs 812), memory 814, some number of input/output (I/O) pins 816, and an analog-to-digital converter (ADC 818). Temperature sensors 806 can each be coupled to an input/output pin 816, and a temperature measurement from the temperature sensor 806 can be converted from a voltage value to a temperature value by the ADC 818.

Memory of the controller 808 can store one or more set of computer-readable instructions which configure the controller 808 to operate at least a first feedback loop by performing a PID computation of one or more first input signals, a first proportional term, a first integral term, a first derivative term, and a first setpoint to output one or more control signals, and a second feedback loop by performing a PID computation of one or more second input signals, a second proportional term, a second integral term, a second derivative term, and a second setpoint to output one or more control signals.

Furthermore, a controller 808 can be configured to perform PID computation so as to avoid integral term windup.

It should be understood that, according to example embodiments of the present disclosure, first input signals and second input signals may not be input signals from a same source, but, conversely, control signals output by PID computing the first feedback loop and by PID computing the second feedback loop may include one or more control signals sent to a same target.

The controller 808 can operate a first feedback loop by PID computing a first input signal, which can be a temperature measurement input signal received from either the temperature sensor 40A or the temperature sensor 40B, along with a first proportional term, a first integral term, a first derivative term, and a first setpoint, outputting, respectively, a power signal for a heating element 6A or a heating element 6B, and outputting a fan signal for a circulation fan 23.

The first proportional term, the first integral term, and the first derivative term can each be configured such that, while a temperature measurement input signal represents a value lower than the first setpoint, the PID computations can result in a power signal sustaining power to a respective heating element (to induce further heat dissipation at a present rate to the spot airflow measured), and can result in a fan signal sustaining power to a circulation fan 23 (to induce fluid shear and mechanical heat at a present rate to the spot airflow measured).

The first proportional term, the first integral term, and the first derivative term can each be configured such that, while a temperature measurement input signal represents a value higher than the first setpoint, the PID computations can result in a power signal reducing power to a respective heating element (to induce further heat dissipation at a reduced rate to the spot airflow measured), and can result in a fan signal reducing power to a circulation fan 23 (to induce fluid shear and mechanical heat at a reduced rate to the spot airflow measured).

However, it should be understood that, by the controller 808 being configured to operate the second feedback loop as described below, a temperature measurement input signal of the first feedback loop is not expected to represent a value higher than the first setpoint, for reasons that shall be described subsequently. The above aspect of the controller 808 operating the first feedback loop is mentioned herein merely for completeness.

The first proportional term, the first integral term, and the first derivative term can each be configured such that, while a temperature measurement input signal represents a value within 1-2° F. of the first setpoint, the PID computations can result in a power signal sustaining, or periodically increasing and decreasing, power to a respective heating element (to induce further heat dissipation at a stably sustained rate, or at a stably oscillating rate, to the spot airflow measured), and can result in a fan signal sustaining, or periodically increasing and decreasing, power to a circulation fan 23 (to induce fluid shear and mechanical heat at a stably sustained rate, or at a stably oscillating rate, to the spot airflow measured).

The controller 808 can be configured to perform computations for the first feedback loop in a substantially similar fashion in either case, with possible differences between a first proportional term, a first integral term, and a first derivative term in each case.

The controller 808 can operate a second feedback loop by PID computing a second input signal, which can be a temperature measurement input signal received from the temperature sensor 41, along with a second proportional term, a second integral term, a second derivative term, and a second setpoint, outputting a power signal for a heating element 6A, a power signal for a heating element 6B, and a fan signal for a circulation fan 23.

The second proportional term, the second integral term, and the second derivative term can each be configured such that, while a temperature measurement input signal represents a value lower than the second setpoint, the PID computations can result in power signals sustaining power to either or both heating elements (to induce further heat dissipation at a present rate to the spot airflow measured), and can result in a fan signal sustaining power to a circulation fan 23 (to induce fluid shear and mechanical heat at a present rate to the spot airflow measured).

The second proportional term, the second integral term, and the second derivative term can each be configured such that, while a temperature measurement input signal represents a value higher than the second setpoint, the PID computations can result in power signals reducing power to either or both heating elements (to induce further heat dissipation at a reduced rate to the spot airflow measured), and can result in a fan signal reducing power to a circulation fan 23 (to induce fluid shear and mechanical heat at a reduced rate to the spot airflow measured).

The second proportional term, the second integral term, and the second derivative term can each be configured such that, while a temperature measurement input signal represents a value within 1-2° F. of the second setpoint, the PID computations can result in power signals sustaining, or periodically increasing and decreasing, power to either or both heating elements (to induce further heat dissipation at a stably sustained rate, or at a stably oscillating rate, to the spot airflow measured), and can result in a fan signal sustaining, or periodically increasing and decreasing, power to a circulation fan 23 (to induce fluid shear and mechanical heat at a stably sustained rate, or at a stably oscillating rate, to the spot airflow measured).

It should be understood that, even though the two different feedback loops result in the controller 808 sending control signals to at least some of the same targets, the first setpoint and the second setpoint are not the same. As described above, the first setpoint can be an offset setpoint which is slightly higher in temperature value than the second setpoint which is an input setpoint; or the second setpoint can be an offset setpoint which is slightly lower in temperature value than the first setpoint which is an input setpoint. In the first case, a user can input a temperature setting, and the controller 808 is configured to set the temperature setting as the second setpoint and set the first setpoint as an offset slightly lower in temperature value than the second setpoint. In the second case, a user can input a temperature setting, and the controller 808 is configured to set the temperature setting as the first setpoint and set the second setpoint as an offset slightly lower in temperature value than the first setpoint.

Therefore, by the controller 808 operating both the first feedback loop and the second feedback loop concurrently, the controller is configured to concurrently: perform a PID computation of at least a first temperature measurement and a first temperature setpoint to send one or more control signals (i.e., a fan signal and a power signal) to the circulation fan and a heating elements to cause the circulation fan and the heating elements to substantially sustain a first spot airflow temperatures (at the temperature sensor 40A downstream from the heating element 6A) at approximately a value of a first temperature setpoint; perform a PID computation of at least a second temperature measurement and a second temperature setpoint to send one or more control signals to the circulation fan and one or both heating elements to cause the circulation fan and the one or both heating elements to substantially sustain a second spot airflow temperature (at a temperature sensor 41 at a sterilization air exhaust port) at approximately a value of a second temperature setpoint, where the second spot airflow temperature to be sustained is slightly lower in temperature value than the first spot airflow temperature to be sustained; and perform a PID computation of at least a third temperature measurement and a first temperature setpoint to send one or more control signals to the circulation fan and a heating element to cause the circulation fan and the heating element to substantially sustain a third spot airflow temperature (at the temperature sensor 40B downstream from the heating element 6B) at approximately a value of a first temperature setpoint.

Furthermore, the controller is configured to send the one or more control signals to further cause the heating element and the circulation fan to sustain a first spot airflow temperature at the first temperature sensor at approximately the value of the first temperature setpoint asymptotically, and/or to further cause the heating element and the circulation fan to sustain a second spot airflow temperature at the second temperature sensor at approximately the value of the second temperature setpoint asymptotically.

According to conventional PID controller implementations, a value of a variable can be sustained at a setpoint asymptotically (i.e., approaching the setpoint value without overshooting), or by stable oscillation (i.e., repeatedly overshooting the setpoint value in both directions). With reference to a dry-heat sterilizer according to example embodiments of the present disclosure, it is advantageous to sustain temperatures at a setpoint asymptotically rather than by oscillation, to avoid overshooting the setpoint temperature and placing heat-intolerant articles and packaging at risk; and to reduce volatility and improve speed in sustaining the temperature at the setpoint. It should be understood that even asymptotic PID controllers may ultimately sustain a value of a variable by some extent of negligible oscillation negligible overshoot; asymptotic PID controller implementations according to example embodiments of the present disclosure, net oscillations of approximately 0.5° F. between a minima and a maxima on either side of a setpoint (and thus overshoot of anywhere from approximately 0.1 to 0.5° F.) can be considered negligible. By way of example, FIG. 7C illustrates such negligible oscillation of sustained spot airflow temperature at the sterilization air exhaust port of FIG. 7B. The vertical axis represents temperature in degrees Fahrenheit, and the horizontal axis represents time in seconds.

Furthermore, it is advantageous to measure and sustain temperatures of different spot airflows along an airflow circuit of the dry-heat sterilizer based on different setpoints, including measuring and sustaining a temperature of at least one or more first spot airflows downstream from a heating assembly along the airflow circuit based on a first setpoint, and measuring and sustaining a temperature of at least a second spot airflow at an exhaust portal of a sterilization chamber, just upstream from a circulation fan which propels the airflow towards a heating assembly based on a second setpoint.

Furthermore, it is advantageous to initialize a first setpoint slightly higher in temperature value than a second setpoint, the differential between the two setpoints reflecting a gradient in loss of heat from the airflow along the direction of the airflow circuit, temperature being approximately highest at one or more first spot airflows downstream from heating assemblies; gradually lowered through an upper supply air duct and a lower supply air duct due to normal radiative heat loss; and further lowered through the sterilization chamber due to heat absorbance by contaminated articles, resulting in temperature being substantially lowest at a second spot airflow at the exhaust portal of the sterilization chamber, just before airflow, propelled by the circulation fan, is returned to the heating assemblies. As described above with reference to Equation 1 and Equation 2, the second spot airflow temperature is lowered as a result of having passed through the sterilization chamber due to heat absorption by articles in the sterilization chamber.

Furthermore, it is advantageous to lower the first setpoint to the value of the second setpoint after the second spot airflow temperature at the exhaust port is sustained at the second setpoint. Initially, the differential between the setpoints configured the dry-heat sterilizer to slightly overheat one or more first spot airflows relative to the second setpoint, without the temperature at the one or more first spot airflows overshooting the first setpoint, the higher rate of heating at the first spot airflows speeding up sustaining, further downstream, the second spot airflow temperature at the second setpoint.

After the first setpoint is lowered to the value of the second setpoint, the controller 808 is configured to begin a sterilization cycle (i.e., a period of sustained heating at the sustained temperature) with the first setpoint and the second setpoint values fixed, sending control signals to the circulation fan and the heating elements to sustain a substantially same temperature at one or more first spot airflows and at the second spot airflow.

Consequently, the controller 808 is configured to operate the second feedback loop to provide some degree of input into the first feedback loop. Before one or more first spot airflow temperatures at the temperature sensors 40A and 40B reach the first setpoint, a second spot airflow temperature at the temperature sensor 41 is already approaching the second setpoint. Then, with second spot airflow temperature sustained at the second setpoint, and before one or more spot airflow temperatures has overshot the first setpoint (where the differential between the two setpoints can be calibrated to minimize the possibility that this would occur), the first setpoint is lowered. Thus, the controller, in sending control signals to the same controlled devices 820, including the same circulation fan and a same heating element, from PID computing two different feedback loops, can avoid outputting contradictory control signals which might have resulted from the two independent PID computations had one or more first spot airflow temperatures overshot the first setpoint before the second spot airflow temperature was sustained at the second setpoint. In this fashion, the second spot airflow temperature can be sustained at the second setpoint asymptotically, and even if the second spot airflow temperature is sustained by stable oscillation at the second setpoint, the likelihood that first spot airflow temperatures at the temperature sensors 40A and 40B will overshoot the first setpoint is substantially reduced. This results in first spot airflow temperatures at the temperature sensors 40A and 40B being sustained at the first setpoint asymptotically, substantially improving precision of heat control cycles in a dry-heat sterilizer according to example embodiments of the present disclosure.

FIG. 9 illustrates an example system 900 for implementing the processes and methods described above for implementing a multi-loop heat control cycle.

The techniques and mechanisms described herein may be implemented by multiple instances of the system 900, as well as by any other computing device, system, and/or environment. The system 900 shown in FIG. 9 is only one example of a system and is not intended to suggest any limitation as to the scope of use or functionality of any computing device utilized to perform the processes and/or procedures described above. Other well-known computing devices, systems, environments and/or configurations that may be suitable for use with the embodiments include, but are not limited to, personal computers, server computers, hand-held or laptop devices, multiprocessor systems, microprocessor-based systems, set top boxes, game consoles, programmable consumer electronics, network PCs, minicomputers, mainframe computers, distributed computing environments that include any of the above systems or devices, implementations using field programmable gate arrays (“FPGAs”) and application specific integrated circuits (“ASICs”), and/or the like.

The system 900 may include one or more processors 902 and system memory 904 communicatively coupled to the processor(s) 902. The processor(s) 902 and system memory 904 may be physical or may be virtualized and/or distributed. The processor(s) 902 may execute one or more modules and/or processes to cause the processor(s) 902 to perform a variety of functions. In embodiments, the processor(s) 902 may include a central processing unit (“CPU”), a graphics processing unit (“GPU”), both CPU and GPU, or other processing units or components known in the art. Additionally, each of the processor(s) 902 may possess its own local memory, which also may store program modules, program data, and/or one or more operating systems.

Depending on the exact configuration and type of the system 900, the system memory 904 may be volatile, such as RAM, non-volatile, such as ROM, flash memory, miniature hard drive, memory card, and the like, or some combination thereof. The system memory 904 may include one or more computer-executable modules 906 that are executable by the processor(s) 902.

The modules 906 may include, but are not limited to, a first PID computing module 908, a second PID computing module 910, an input signal receiving module 912, a control signal outputting module 914, and a setpoint setting module 916.

The first PID computing module 908 can configure one or more processors to PID compute a first feedback loop as described above with reference to FIG. 8.

The second PID computing module 910 can configure one or more processors to PID compute a second feedback loop as described above with reference to FIG. 8.

The input signal receiving module 912 can configure one or more processors to receive one or more input signals as described above with reference to FIG. 8.

The control signal outputting module 914 can configure one or more processors to send one or more control signals, including at least a power signal and a fan signal, as described above with reference to FIG. 8.

The setpoint setting module 916 can configure one or more processors to set one or more setpoints as described above with reference to FIG. 8.

The system 900 may additionally include an input/output (I/O) interface 940 and a communication module 950 allowing the system 900 to communicate with other systems and devices over a network. The network may include the Internet, wired media such as a wired network or direct-wired connections, and wireless media such as acoustic, radio frequency (“RF”), infrared, and other wireless media.

Some or all operations of the methods described above can be performed by execution of computer-readable instructions stored on a computer-readable storage medium, as defined below. The term “computer-readable instructions” as used in the description and claims, include routines, applications, application modules, program modules, programs, components, data structures, algorithms, and the like. Computer-readable instructions can be implemented on various system configurations, including single-processor or multiprocessor systems, minicomputers, mainframe computers, personal computers, hand-held computing devices, microprocessor-based, programmable consumer electronics, combinations thereof, and the like.

The computer-readable storage media may include volatile memory (such as random-access memory (“RAM”)) and/or non-volatile memory (such as read-only memory (“ROM”), flash memory, etc.). The computer-readable storage media may also include additional removable storage and/or non-removable storage including, but not limited to, flash memory, magnetic storage, optical storage, and/or tape storage that may provide non-volatile storage of computer-readable instructions, data structures, program modules, and the like.

A non-transient computer-readable storage medium is an example of computer-readable media. Computer-readable media includes at least two types of computer-readable media, namely computer-readable storage media and communications media. Computer-readable storage media includes volatile and non-volatile, removable and non-removable media implemented in any process or technology for storage of information such as computer-readable instructions, data structures, program modules, or other data. Computer-readable storage media includes, but is not limited to, phase change memory (“PRAM”), static random-access memory (“SRAM”), dynamic random-access memory (“DRAM”), other types of random-access memory (“RAM”), read-only memory (“ROM”), electrically erasable programmable read-only memory (“EEPROM”), flash memory or other memory technology, compact disk read-only memory (“CD-ROM”), digital versatile disks (“DVD”) or other optical storage, magnetic cassettes, magnetic tape, magnetic disk storage or other magnetic storage devices, or any other non-transmission medium that can be used to store information for access by a computing device. In contrast, communication media may embody computer-readable instructions, data structures, program modules, or other data in a modulated data signal, such as a carrier wave, or other transmission mechanism. As defined herein, computer-readable storage media do not include communication media.

The computer-readable instructions stored on one or more non-transitory computer-readable storage media that, when executed by one or more processors, may perform operations described above with reference to FIGS. 1 to 8. Generally, computer-readable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes.

Although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A method comprising:
performing a proportional-integral-derivative (PID) computation of a first temperature measurement input signal and a first temperature setpoint to output one or more control signals, and
performing a PID computation of a second temperature measurement input signal and a second temperature setpoint to output one or more control signals;
wherein the first input signal is received from a first temperature sensor and the second input signal is received from a second temperature sensor, and
wherein the one or more control signals target at least a first heating element from which the first temperature sensor is downstream in an airflow circuit, and a circulation fan from which the second temperature sensor is upstream in the airflow circuit.

2. The method of claim 1, wherein the one or more control signals cause the first heating element and the circulation fan to sustain a first spot airflow temperature at the first temperature sensor at approximately a value of the first temperature setpoint and cause one or both of the first heating element and a second heating element and the circulation fan to sustain a second spot airflow temperature at the second temperature sensor at approximately a value of the second temperature setpoint.

3. The method of claim 2, wherein the one or more control signals further cause the first heating element and the circulation fan to sustain a first spot airflow temperature at the first temperature sensor at approximately the value of the first temperature setpoint asymptotically.

4. The method of claim 1, wherein the first setpoint is slightly higher in temperature value than the second setpoint.

5. The method of claim 1, further comprising performing a PID computation of a third temperature measurement input signal and the first temperature setpoint to output one or more control signals;
wherein the third input signal is received from a third temperature sensor, and
wherein the one or more control signals target at least a second heating element from which the third temperature sensor is downstream in the airflow circuit.

6. The method of claim 5, wherein the airflow circuit passes through a sterilization chamber after passing the first and third temperature sensors and before reaching the second temperature sensor.

7. The method of claim 5, wherein the first heating element and the second heating element each comprises an openwork heating element.

8. A system comprising:
one or more processors; and
memory communicatively coupled to the one or more processors, the memory storing computer-executable modules executable by the one or more processors that, when executed by the one or more processors, perform associated operations, the computer-executable modules comprising:
a first PID computing module executable by the one or more processors to perform a proportional-integral-derivative (PID) computation of a first temperature measurement input signal and a first temperature setpoint to cause a control signal outputting module to output one or more control signals, and
a second PID computing module executable by the one or more processors to perform a PID computation of a second temperature measurement input signal and a second temperature setpoint to cause the control signal outputting module to output one or more control signals;
an input signal receiving module executable by the one or more processors to receive the first input signal from a first temperature sensor and receive the second input signal from a second temperature sensor, and
a control signal outputting module executable by the one or more processors to output the one or more control signals targeting at least a heating element from which the first temperature sensor is downstream in an airflow circuit, and a circulation fan from which the second temperature sensor is upstream in the airflow circuit.

9. The system of claim 8, wherein the one or more control signals cause the first heating element and the circulation fan to sustain a first spot airflow temperature at the first temperature sensor at approximately a value of the first temperature setpoint and cause one or both of the first heating element and a second heating element and the circulation fan to sustain a second spot airflow temperature at the second temperature sensor at approximately a value of the second temperature setpoint.

10. The system of claim 9, wherein the one or more control signals further cause the first heating element and the circulation fan to sustain a first spot airflow temperature at the first temperature sensor at approximately the value of the first temperature setpoint asymptotically.

11. The system of claim 8, wherein the first setpoint is slightly higher in temperature value than the second setpoint.

12. The system of claim 8, wherein the first PID computing module is further executable by the one or more processors to perform a PID computation of a third temperature measurement input signal and the first temperature setpoint to cause the control signal outputting module to output one or more control signals;
wherein the input signal receiving module is further executable by the one or more processors to receive a third input signal from a third temperature sensor, and
wherein the control signal outputting module is further executable by the one or more processors to output the one or more control signals targeting at least a second heating element from which the third temperature sensor is downstream in the airflow circuit.

13. The system of claim 12, wherein the airflow circuit passes through a sterilization chamber after passing the first and third temperature sensors and before reaching the second temperature sensor.

14. The system of claim 12, wherein the first heating element and the second heating element each comprises an openwork heating element.

15. A sterilizer, comprising:
a sterilization chamber; and
a supply air duct connected to the sterilization chamber by an exhaust portal, the supply air duct comprising an upper branch and a lower branch each connected to an entry portal of the sterilization chamber;
wherein the upper branch and the lower branch respectively contain an upper heating assembly and a lower heating assembly, each heating assembly comprising an openwork heating element.

16. The sterilizer of claim 15, further comprising:
a first temperature sensor and a second temperature sensor respectively downstream in the airflow circuit from the upper heating assembly and from the lower heating assembly; and
a third temperature sensor at the exhaust portal.

17. The sterilizer of claim 15, wherein each openwork heating element comprises a coiled-coil heating element mounted across an electrical insulator spanning a cross-sectional width of the upper branch or the lower branch.

18. The sterilizer of claim 17, wherein a surface of each electrical insulator comprises coiled grooves wherein a coiled-coil heating element is mounted and held in place.

19. The sterilizer of claim 15, wherein each openwork heating element comprises a zigzag heating element spanning a cross-sectional width of the upper branch or the lower branch.

20. The sterilizer of claim 15, wherein the openwork heating element comprises a coiled heating element mounted across an electrical insulator spanning a cross-sectional width of the upper branch and the lower branch, wherein airflow is permitted between each first-order coil of the heating element and the electrical insulator.
